(19) United States
(12) Patent Application Publication
(10) Pub. No.: US 2007/0093707 A1
(43) Pub. Date: Apr. 26, 2007

(54) Biological signal transmitter and biological-information sensor

(76) Inventor: Eriko Noguchi, Chiba-shi (JP)

Correspondence Address: BRUCE L. ADAMS, ESQ.;SUITE 1231, 17 BATTERY PLACE, NEW YORK, NY, 10004, US

(21) Appl. No.: 11/583,181

(22) Filed: Oct. 19, 2006

(30) Foreign Application Priority Data
Oct. 25, 2005 (JP) 2005-309551

(51) Int. Cl. A61B 5/04

(52) U.S. Cl. 600/390; 600/393; 128/903

(57) ABSTRACT

To prevent the operation start switch of a biological signal transmitter from being turned on even if the pair of breast electrodes is short-circuited in nonuse. A breast electrode is mounted to the vicinity of one end of a belt to which one end of a casing of a biological signal transmitter is secured. A breast electrode is mounted to the vicinity of the other end of the belt to which a fixed part of a belt joint having a fixed portion is secured. The breast electrode and a circuit board are connected to the circuit board with a sensor signal line. The breast electrode and the circuit board are connected to each other with a sensor signal line formed of a fixing-part-side conducting path, a fixing-part-side contact, a fixed-part-side contact, and a fixed-part-side conducting path. The belt joint constitutes an on-off switch that is operated according to the engagement and disengagement of the fixing part and the fixed part. The on-off switch is used to constitute an attachment sensor circuit.

### BACKGROUND OF THE INVENTION

1. Field of the Invention

The present invention relates to a biological signal transmitter and a biological-information sensor having the same. A typical example of the biological-information sensor is a pulse rate meter including a heart-rate measuring transmitter having a pair of breast electrodes and a breast belt and a watch heart-rate measuring receiver.

2. Description of the Prior Art

As shown in FIG. 8, a heart-rate measuring transmitter 10 generally includes a pair of breast electrodes 2 and 3 mounted to a breast belt; a signal operating circuit 11 that operates heart-rate data per unit time on the basis of an electrocardiographic signal sensed by the breast electrodes 2 and 3 and outputs it; a wireless transmitting circuit 12 that converts the heart-rate data to a radio signal and outputs it via a transmission antenna; an attachment detection circuit 13 that detects attachment to a human body; and a power control circuit 14.

The power control circuit 14 has a button cell as power source, and starts heart-rate measurement in response to the signal from the attachment detection circuit 13. The attachment sensing electrodes of the attachment detection circuit 13 are used both as the pair of breast electrodes 2 and 3. The power control circuit 14 controls the power of the signal operating circuit 11 and the wireless transmitting circuit 12 according to the resistance of a human body measured between the pair of breast electrodes 2 and 3 attached to the human body.

Conventional biological signal transmitters having a breast belt include one whose body case is integrated with a breast belt, as disclosed in JP-A-6-245913, and one having a detachable biological signal transmitter unit and a breast belt having an opening for the unit, the biological signal transmitter unit being fitted in the opening for the unit, as disclosed in JP-A-2002-143110.

In the case of the conventional heart-rate measuring transmitters, when a user wears the breast belt on the breast, the pair of breast electrodes comes in close contact with the body to generate an attachment detection signal in the attachment detection circuit. Then, the power control circuit starts heart-rate measurement in response to the attachment detection signal. Briefly, the conventional heart-rate measuring transmitters are constructed such that when attached onto the body of a user, driving current is supplied to the various electronic circuits of the heart-rate measuring transmitters. In other words, the pair of breast electrodes works as a power switch for the heart-rate measuring transmitter.

As has been described, the conventional heart-rate measuring transmitters including a pair of breast electrodes and a breast belt has a structure in which the pair of breast electrodes functions as a power switch. Therefore, the heart-rate measuring transmitter disclosed in JP-A-6-245913 has the problem that when the user removes the breast belt and places it on a steel desk, the power switch is turned on by the contact of the breast electrodes with the surface of the desk, resulting in wasting of power. In other words, with the heart-rate measuring transmitter disclosed in JP-A-6-245913, the pair of breast electrodes is short-circuited in nonuse by a conductive object such as a steel desk.

The heart-rate measuring transmitter disclosed in JP-A-2002-143110 has the problem that, to prevent waste of power, the biological signal transmitter unit has to be removed from the breast belt every time the user removes the breast belt and places it on a steel desk. However, since the opening for the unit accommodates the biological signal transmitter unit completely to improve moisture resistance, it is very troublesome to remove and mount the biological signal transmitter unit from and to the breast belt. Thus also the heart-rate measuring transmitter of JP-A-2002-143110 has the problem that when the biological signal transmitter unit that is not removed from the breast belt is placed on a steel desk, and thus the power switch is turned on by the contact of the breast electrodes with the surface of the desk, resulting in waste of power. In short, also with the heart-rate measuring transmitter disclosed in JP-A-2002-143110, the pair of breast electrodes is short-circuited in nonuse by a conductive object such as a steel desk.

An object of the invention is to prevent the operation start switch of a biological signal transmitter having a pair of breast electrodes and a breast belt from being turned on even if the pair of breast electrodes is short-circuited in nonuse.

### SUMMARY OF THE INVENTION

In order to solve the above-described problem, a biological signal transmitter is provided which includes: a first breast electrode and a second breast electrode; a signal operating circuit that performs a specified process on a biological signal detected by the breast electrodes: a wireless transmitting circuit that transmits the output signal from the signal operating circuit by radio; an attachment detection circuit that uses the first breast electrode and the second breast electrode both as an attachment sensor; and a power control circuit, the biological signal transmitter being mounted to a breast belt.

A fixing part and a fixed part of a belt joint of the breast belt each have a conducting path to constitute an on-off switch that is operated according to the engagement and disengagement of the fixing part and the fixed part, and an attachment sensor circuit is constituted using the on-off switch.

### BRIEF DESCRIPTION OF THE SEVERAL VIEWS OF THE DRAWINGS

A preferred form of the present invention is illustrated in the accompanying drawings in which:

FIG. 1 is a longitudinal sectional view of a biological signal transmitter according to a first embodiment of the invention, wherein the fixing part and the fixed part of the breast belt are joined to each other;

FIG. 2 is a longitudinal plan view of the biological signal transmitter of the first embodiment, wherein the fixing part and the fixed part of the breast belt are separated from each other;

FIG. 3 is a longitudinal plan view of the biological signal transmitter of the first embodiment, wherein the fixing, part and the fixed part of the breast belt are joined to each other;

FIG. 4 is a perspective view of the fixing part and the fixed part of the breast belt employed in the first embodiment;

FIG. 5 is a block circuit diagram of the biological signal transmitter of the first embodiment;

FIG. 6 is a block circuit diagram of a biological signal transmitter of a second embodiment of the invention;

FIG. 7 is a block circuit diagram of a biological signal transmitter of a third embodiment of the invention; and

FIG. 8 is a block circuit diagram of a conventional biological signal transmitter.

### DETAILED DESCRIPTION OF THE PREFERRED EMBODIMENTS

A biological signal transmitter according to the invention includes: a first breast electrode and a second breast electrode; a signal operating circuit that performs a specified process on a biological signal detected by the breast electrodes: a wireless transmitting circuit that transmits the output signal from the signal operating circuit by radio; an attachment detection circuit that uses the first breast electrode and the second breast electrode both as an attachment sensor; and a power control circuit, the biological signal transmitter being mounted to a breast belt.

A fixing part and a fixed part of a belt joint of the breast belt each have a conducting path to constitute an on-off switch that is operated according to the engagement and disengagement of the fixing part and the fixed part. The on-off switch is placed in the middle of one of sensor signal lines connecting the first breast electrode, the second breast electrode, and the circuit board of the biological signal transmitter to constitute an attachment sensor circuit by the first breast electrode, the second breast electrode, and the on-off switch.

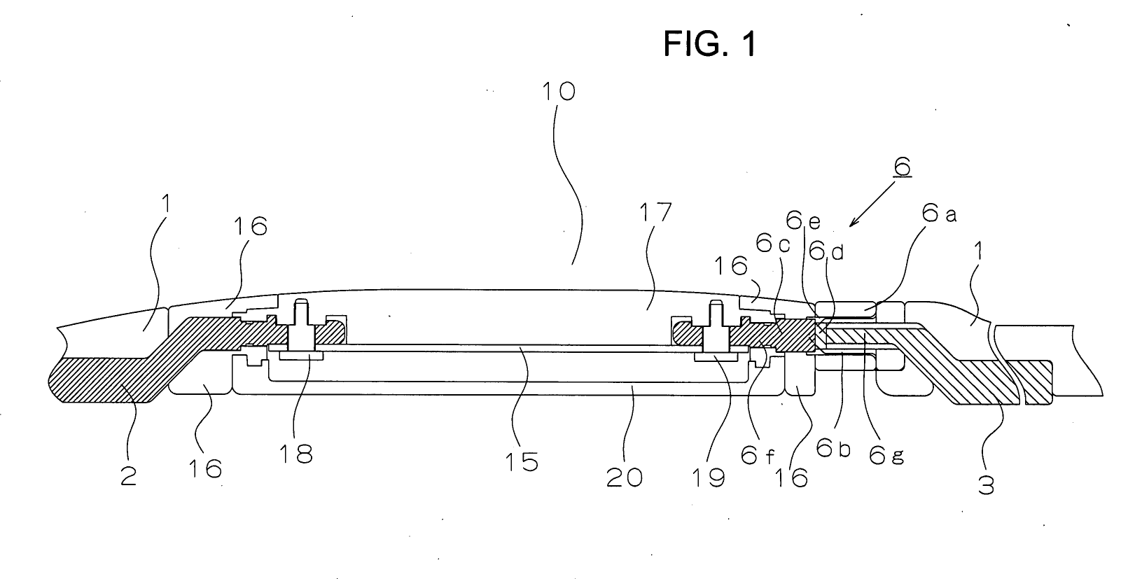

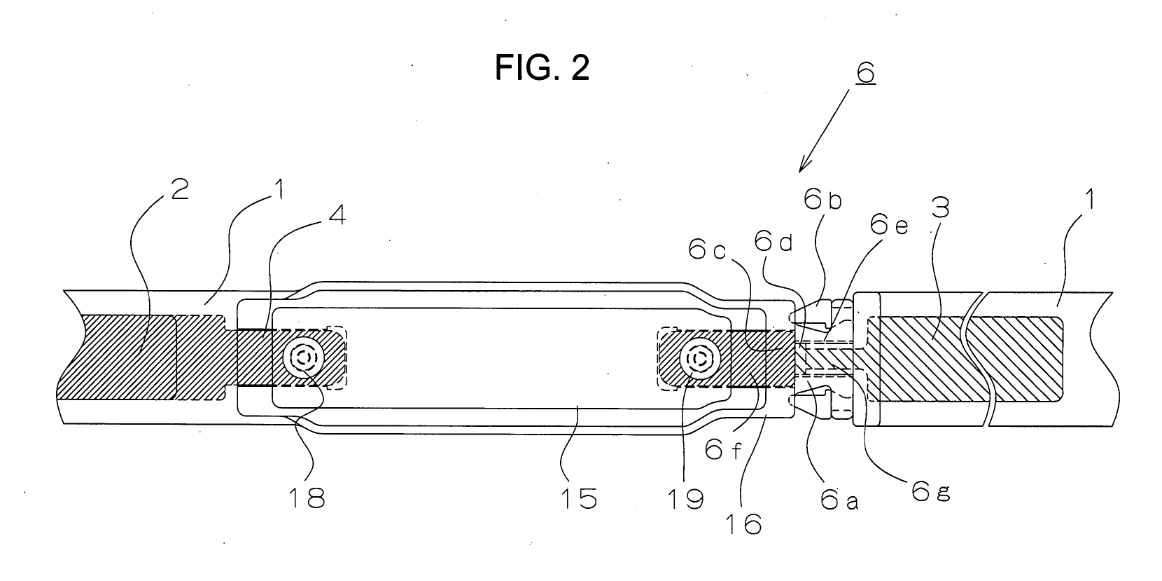

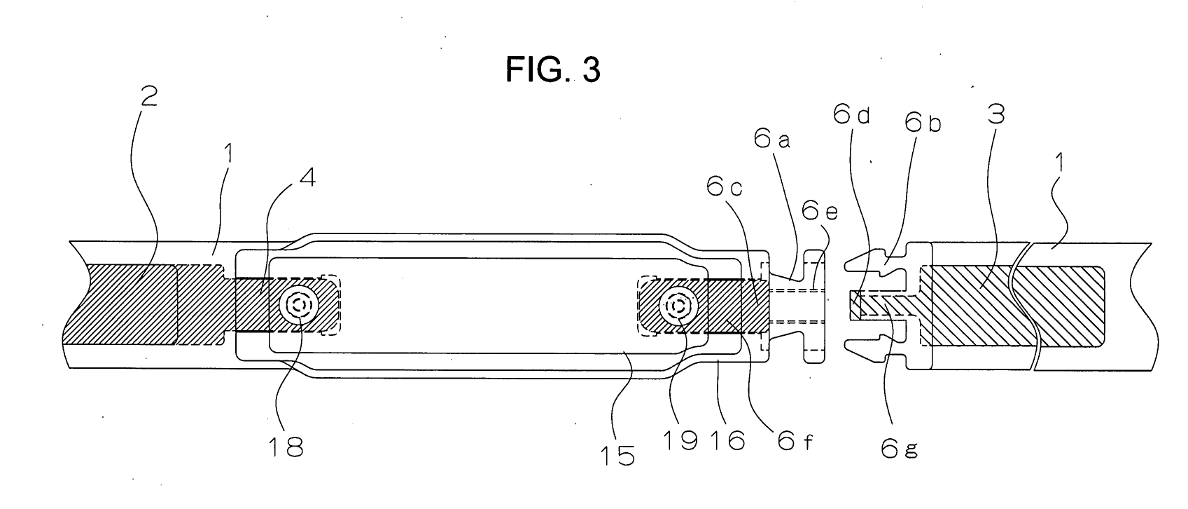

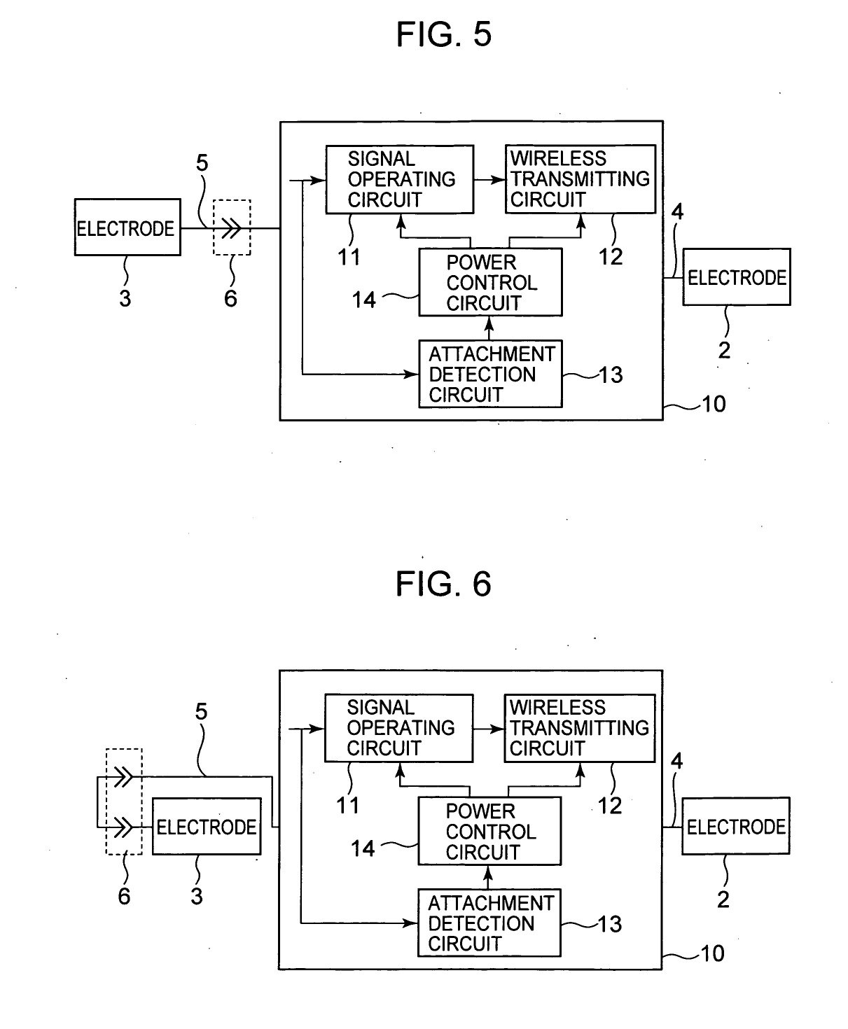

As shown in FIGS. 1 to 5, a biological signal transmitter 10 according to a first embodiment of the invention includes: a first breast electrode 2 and a second breast electrode 3; a signal operating circuit 11 that performs a predetermined process on the biological signal detected by the breast electrodes; a wireless transmitting circuit 12 that transmits the output signal of the signal operating circuit 11 by radio; an attachment detection circuit 13 that uses the first and second breast electrodes 2 and 3 both as an attachment sensor; and a power control circuit 14. A belt joint 6 of the breast belt. 1 has a fixing part and a fixed part having conducting paths 6f and 6g, respectively, to construct an on-off switch that is operated according to the engagement and disengagement of the fixing part and the fixed part. Thus an attachment sensor circuit is configured by the on-off switch.

Specifically, the biological signal transmitter 10 of the first embodiment is constructed such that a casing 16 whose one end is secured to one end of the belt 1 accommodates a circuit board 15, whose surface is closed by a frame 17, and whose back is closed with a back cover 20. The circuit board 15 has the signal operating circuit 11, the wireless transmitting circuit 12, the attachment detection circuit 13, and the power control circuit 14 formed thereon. The casing 16 is of an elongated substantially rectangular casing in a flat shape with a proportion of about 1 to 4 and a width a little wider than that of the belt 1.

The breast electrode 2 of the pair of breast electrodes is fixed to a first end of the belt to which one end of the casing 16 of the biological signal transmitter 10 is secured. The breast electrode 2 and the circuit board 15 are connected together with a sensor signal line 4. As shown in FIGS. 1 to 3, the sensor signal line 4 is formed of a metal plate integrated with the breast electrode 2. One end of the sensor signal line 4 is connected to a specified portion of the printed circuit of the circuit board 15 with pins 18 and 19.

A second end of the casing 16 is secured to a fixing part of the belt joint 6 having a fixing portion 6a. The fixed part of the belt joint 6 having fixed portions 6b is secured to the second end of the belt 1.

The breast electrode 3 of the pair of breast electrodes is fixed to the vicinity of the second end of the belt 1 to which fixed part of the belt joint 6 having the fixed portions 6b is secured. The breast electrode 3 and the circuit board 15 are connected together with a sensor signal line 5. As shown in FIGS. 1 to 3, the sensor signal line 5 is formed of a fixing-part-side conducting path 6f, a fixing-part-side contact 6c, a fixed-part-side contact 6d, and a fixed-part-side conducting path 6g. The fixed-part-side conducting path 6g is formed of a metal plate integrated with the breast electrode 3, one end of which connects to the fixed-part-side contact 6d. The fixing-part-side conducting path 6f is formed of the same metal plate as the breast electrode 3, a first end of which connects to the fixing-part-side contact 6c, while a second end of which connects to a specified portion of the printed circuit of the circuit board 15 with a pin 19.

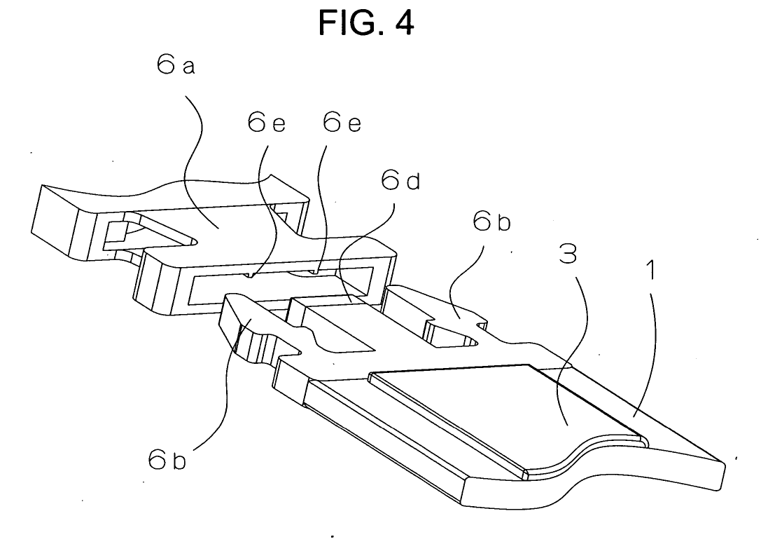

As shown in the perspective of FIG. 4, the belt joint 6 is composed of a fixing part having the fixing portion 6a; and a fixed part having one contact projection and the two fixed portions 6b. The contact projection is a rectangular-section projection formed in the center of the end of the fixed part, and has the fixed-part-side contact-6d at the end face. The two fixed portions 6b are mushroom-shaped projections formed at the right and left of the end of the fixed part of the contact projection.

The fixing portion 6a of the fixing part of the belt joint 6 has a rectangular-section front through hole of a sufficient size to engage the fixed part having one contact projection and the two fixed portions 6b, and a depth through which the heads of the mushroom-shaped fixed portions 6b pass. The fixing portion 6a further has a rear-side engaging hole and upper and lower connecting plates between the rectangular-section through hole and the casing 16 of the biological signal transmitter 10. The rear-side engaging hole is a rectangular-section opening formed at the casing 16 of the biological signal transmitter 10. The fixing-part-side contact 6c is formed on the surface of the inner part of the rear-side engaging hole. The rectangular-section front through hole has two rail-shaped projections 6e on the upper inner surface thereof. The interval between the two rail-shaped projections 6e is suited to guiding the rectangular-section projection formed in the center of the end of the fixed part of the belt joint 6.

When the fixing part and the fixed part of the belt joint 6 with the above-described structure are joined together, the fixing-part-side contact 6c and the fixed-part-side contact 6d come into contact and are held in this state. Then, the sensor signal line 5 between the breast electrode 3 and the circuit board 15 of the biological signal transmitter 10 is brought into conduction and is held in this state. Thus, the breast electrode 2 and the sensor signal line 4, the breast electrode 3 and the sensor signal line 5, and the biological signal transmitter 10 mounted to the body of the user with the belt 1 are connected to form an attachment sensor circuit. The attachment detection circuit 13 senses the current from the attachment sensor circuit. Then the power control circuit 14 starts the operation of the signal operating circuit 11 and the wireless transmitting circuit 12 in response to the output signal from the attachment detection circuit 13.

In other words, the biological signal transmitter of the first embodiment is characterized in that the first breast electrode 2 is mounted to one end of the breast belt 1, while the second breast electrode 3 is mounted to the other end of the breast belt 1; and the on-off switch is placed in the middle of the sensor signal line 5 that connects the second breast electrode 3 with the circuit board 15 of the biological signal transmitter 10, thus configuring a attachment sensor circuit by the first breast electrode 2, the second breast electrode 3, and the on-off switch.

The fixing part and the fixed part of the belt joint 6 have a temporarily engaging structure, as shown in the perspective view of FIG. 4. This enables to maintain a no-current state of the biological signal transmitter without removing the breast belt 1 while biological-signal detection is not performed. Furthermore, the on-off switch at the belt joint of the breast belt can provide stable conduction because the insertion hole of the fixed portion is guided to the contact, and there is no need to increase the size because of the provision of the contact at the end of the rails.

As shown in FIG. 6, the biological signal transmitter 10 according to a second embodiment of the invention is characterized in that the first breast electrode 2 and the second breast electrode 3 are mounted to one end of the breast belt 1, and the on-off switch of the first embodiment is placed in the middle of the sensor signal line 5 that connects the second breast electrode 3 with the 15 of the biological signal transmitter, thus configuring an attachment sensor circuit by the first breast electrode 2, the second breast electrode 3, and the on-off switch.

The biological signal transmitter 10 of the second embodiment is constructed such that all of the casing 16 thereof, the first breast electrode 2, and the second breast electrode 3 are mounted to one end of the breast belt 1. Thus, only the sensor signal line 5 is disposed at the other end of the breast belt 1. The sensor signal line 5 extending from the casing 16 of the biological signal transmitter 10 to the other end of the breast belt 1 via the belt joint 6 is turned back. The turned-back sensor signal line 5 extends to the casing 16 via the belt joint 6.

Since the sensor signal line 5 is turned back, the on-off switch provided at the belt joint 6 needs to be partly modified from that of the first embodiment. The on-off-switch of the first embodiment is constructed to connect or disconnect one conducting path, while that of the second embodiment connects or disconnects two conducting paths.

While the belt joint 6 of the first embodiment is disposed in the vicinity of the biological signal transmitter 10, that is, at the front of a human body, as shown in FIGS. 1 to 4, the belt joint 6 of the second embodiment may be disposed on the side of the human body.

The biological signal transmitter 10 according to a third embodiment of the invention includes: the first breast electrode 2 and the second breast electrode 3; the signal operating circuit 11; the wireless transmitting circuit 12; the attachment detection circuit 13 that detect attachment to a human body; and the power control circuit 14. The biological signal transmitter 10 mounted to the breast belt 1 has a conducting path at each of the fixing part and the fixed part of the belt joint 6 of the breast belt 1 to constitute an on-off switch that is operated according to the engagement and disengagement of the fixing part and the fixed part. Thus, an attachment-sensor signal line 7 of the attachment detection circuit 13 and the on-off switch constitute an attachment sensor circuit.

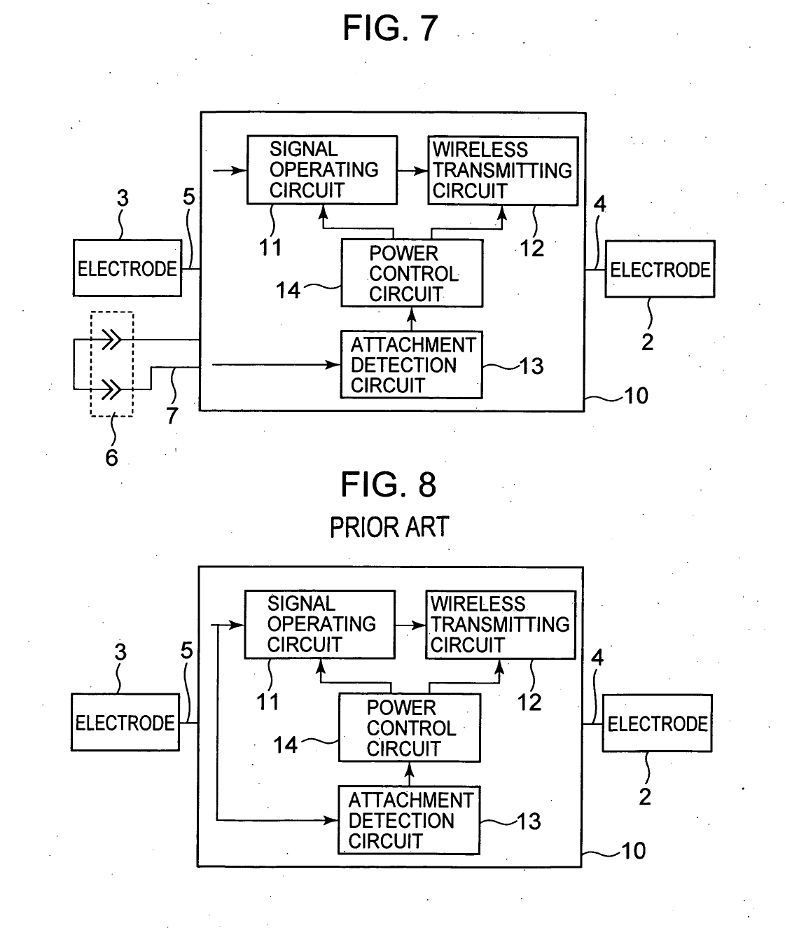

As shown in FIG. 7, the biological signal transmitter 10 of the third embodiment is constructed such that all of the casing 16 thereof, the first breast electrode 2, and the second breast electrode 3 are mounted to one end of the breast belt 1. Thus, only the attachment-sensor signal line 7 is disposed at the other end of the breast belt 1. The attachment-sensor signal line 7 extending from the casing 16 of the biological signal transmitter 10 to the other end of the breast belt 1 via the belt joint 6 is turned back. The turned-back attachment-sensor signal line 7 extends to the casing 16 via the belt joint 6.

Since the attachment-sensor signal line 7 is turned back, the on-off switch provided at the belt joint 6 needs to be partly modified from that of the first embodiment. The on-off switch of the first embodiment is constructed to connect or disconnect one conducting path, while that of the second embodiment connects or disconnects two conducting paths.

While the belt joint 6 of the first embodiment is disposed in the vicinity of the biological signal transmitter 10, that is, at the front of a human body, as shown in FIGS. 1 to 4, the belt joint 6 of the third embodiment in which the pair of breast electrodes 2 and 3 is not used both as an attachment sensor may be disposed on the side or back of the human body.

The invention enables to prevent the operation start switch of a biological signal transmitter from being turned on even if the pair of breast electrodes is short-circuited in nonuse. This increases the life of the cell in comparison with conventional biological signal transmitters.

According to the invention, the belt joint of the breast belt is disposed on the breast of the user. This facilitates the attachment and detachment of the biological signal transmitter to and from the body in comparison with the conventional biological signal transmitters having a breast belt disposed on the back of the user.

Furthermore, the on-off switch provided at the belt joint of the breast belt is constructed such that the fixed-part insertion hole is guided to the contact by rails. This offers the features that stable conduction is provided and that provision of contact at the ends of the rails eliminates the need for increasing the size.

Furthermore, since a biological signal transmitter according to the invention having a temporary holder at the on-off switch of the belt joint of the breast belt is not removed from the body while biological-signal detection is not executed, a no-current state can be maintained.

What is claimed is:

1. A biological signal transmitter comprising: a first breast electrode and a second breast electrode; a signal operating circuit that performs a specified process on a biological signal detected by the breast electrodes: a wireless transmitting circuit that transmits the output signal from the signal operating circuit by radio; an attachment detection circuit that uses the first breast electrode and the second breast electrode both as an attachment sensor; and a power control circuit, the biological signal transmitter being mounted to a breast belt, wherein
a fixing part and a fixed part of a belt joint of the breast belt each have a conducting path to constitute an on-off switch that is operated according to the engagement and disengagement of the fixing part and the fixed part, and an attachment sensor circuit is constituted by the on-off switch.

2. A biological signal transmitter according to claim 1, wherein the on-off switch is placed in the middle of one of sensor signal lines connecting the first breast electrode, the second breast electrode, and the circuit board of the biological signal transmitter to constitute an attachment sensor circuit by the first breast electrode, the second breast electrode, and the on-off switch.

3. A biological signal transmitter according to claim 1, wherein the first breast electrode is mounted to one end of the breast belt, the second breast electrode is mounted to the other end of the breast belt, and the on-off switch is placed in the middle of a sensor signal line connecting the second breast electrode and the circuit board of the biological signal transmitter to constitute an attachment sensor circuit by the first breast electrode, the second breast electrode, and the on-off switch.

4. A biological signal transmitter according to claim 1, wherein the first breast electrode and the second breast electrode are mounted to one end of the belt, and the on-off switch is placed in the middle of one of sensor signal lines connecting the first breast electrode, the second breast electrode, and the circuit board of the biological signal transmitter to constitute an attachment sensor circuit by the first breast electrode, the second breast electrode, and the on-off switch.

5. A biological signal transmitter comprising: a first breast electrode and a second breast electrode; a signal operating circuit that performs a specified process on a biological signal detected by the breast electrodes: a wireless transmitting circuit that transmits the output signal from the signal operating circuit by radio; an attachment detection circuit that detects attachment to a human body; and a power control circuit, the biological signal transmitter being mounted to a breast belt, wherein
a fixing part and a fixed part of a belt joint of the breast belt each have a conducting path to constitute an on-off switch that is operated according to the engagement and disengagement of the fixing part and the fixed part, and an attachment sensor circuit is constituted by an attachment-sensor signal line of the attachment detection circuit and the on-off switch.
